(19) United States
(12) Patent Application Publication
(10) Pub. No.: US 2022/0288037 A1
(43) Pub. Date: Sep. 15, 2022

(54) PRODRUGS AND FORMULATIONS THEREOF

(71) Applicant: BOARD OF REGENTS OF THE UNIVERSITY OF NEBRASKA, Lincoln, NE (US)

(72) Inventors: Benson Edagwa, Omaha, NE (US); Howard Gendelman, Omaha, NE (US)

(21) Appl. No.: 17/631,955

(22) PCT Filed: Aug. 21, 2020
(86) PCT No.: PCT/US2020/047329

§ 371 Date: Feb. 1, 2022

Related U.S. Application Data
(60) Provisional application No. 62/890,194, filed on Aug. 22, 2019.

(51) Int. Cl. A61K 31/426; A61P 31/12; A61K 31/675

(52) U.S. Cl. CPC A61K 31/426; A61P 31/12; A61K 31/675

(57) ABSTRACT

The present invention provides prodrugs and methods of use thereof.

This application claims priority under 35 U.S.C. § 119(e) to U.S. Provisional Patent Application No. 62/890,194, filed Aug. 22, 2019. The foregoing application is incorporated by reference herein.

This invention was made with government support under Grants Nos. R01 MH104147, P01 DA028555, R01 NS034239, R01 NS036126, P01 NS031492, P01 MH064570, P30 MH062261, P30 AI078498, R01 AG043540, and R56 AI138613 awarded by the National Institutes of Health. The government has certain rights in the invention.

### FIELD OF THE INVENTION

The present invention relates generally to the delivery of therapeutics. More specifically, the present invention relates to compositions and methods for the delivery of therapeutic agents to a patient for the treatment of a disease or disorder.

### BACKGROUND OF THE INVENTION

Remarkable progress has been made in the development of effective diagnostics and treatments against a number of human pathogens. However, treatment fatigue, lack of financial and social support, co-existing mental symptoms, and/or substance abuse can result in the failure to adhere to critical drug regimens. Long-acting drugs can reduce viral transmission, prevent new infection, affect regimen adherence and limit the emergence of drug resistance and systemic toxicities. Reducing the treatment schedule from daily to monthly or even less-frequent administration provides greater patient privacy and satisfaction and improves regimen adherence. However, only a few drugs have been successfully reformulated into long acting formulations. Accordingly, it is clear that improved long term delivery of drugs is needed.

### SUMMARY OF THE INVENTION

In accordance with the instant invention, prodrugs of thiazolides are provided. In some embodiments, the prodrug is a dimer of a thiazolide connected by a linker (e.g., an optionally substituted aliphatic or alkyl group). In some embodiments, the prodrug comprises a thiazolide modified with an ester moiety (e.g., at the 2-position of the benzene) comprising a hydrophobic and/or lipophilic moiety. In certain embodiments, the hydrophobic and/or lipophilic moiety is an aliphatic or alkyl group. In a particular embodiment, the aliphatic or alkyl group is the alkyl chain of a fatty acid or a saturated linear aliphatic chain, optionally substituted with at least one heteroatom. Compositions comprising at least one prodrug of the instant invention and at least one pharmaceutically acceptable carrier are also encompassed by the present invention.

In accordance with another aspect of the instant invention, nanoparticles comprising at least one prodrug of the instant invention and at least one polymer or surfactant are provided. In a particular embodiment, the prodrug is crystalline. In a particular embodiment, the polymer or surfactant is an amphiphilic block copolymer such as an amphiphilic block copolymer comprising at least one block of poly(oxyethylene) and at least one block of poly(oxypropylene) (e.g., poloxamer 407). The nanoparticle may comprise a polymer or surfactant linked to at least one targeting ligand. An individual nanoparticle may comprise targeted and non-targeted surfactants. In a particular embodiment, the nanoparticles have a diameter of about 100 nm to 1 μm. Compositions comprising at least one nanoparticle of the instant invention and at least one pharmaceutically acceptable carrier are also encompassed by the present invention.

In accordance with another aspect of the instant invention, methods for treating, inhibiting, and/or preventing a disease or disorder in a subject in need thereof are provided. The methods comprise administering to the subject at least one prodrug or nanoparticle of the instant invention, optionally within a composition comprising a pharmaceutically acceptable carrier. In a particular embodiment, the disease or disorder is a viral infection (e.g., a hepatitis infection (e.g., HBV) or coronavirus infection (e.g., severe acute respiratory syndrome coronavirus 2 (SARS-CoV-2; COVID 19)). In a particular embodiment, the method further comprises administering at least one further therapeutic agent or therapy for the disease or disorder, e.g., at least one additional anti-HBV compound or anti-coronavirus compound (e.g., anti-SARS-CoV-2 (COVID 19) compound).

### BRIEF DESCRIPTIONS OF THE DRAWING

FIGS. 1A-1D provide characterization of a prodrug of nitazoxanide (M1NTZ) and a nanoformulation thereof (NM1NTZ). FIG. 1A provides a Fourier transform infrared (FT-IR) spectrum of M1NTZ showing absorption bands at 2915 cm⁻¹ and 2850 cm⁻¹, thereby confirming formation of the prodrug as well as the nuclear magnetic resonance spectroscopy of the compound. FIG. 1B provides a graph of the aqueous solubility of M1NTZ. The prodrug exhibited decreased water solubility. FIG. 1C provides a graph of cell viability as evaluated by mitochondrial function in monocyte-derived macrophage (MDM) by a 3-(4,5-dimethylthiazol-2-yl)-2,5-diphenyltetrazolium bromide (MTT) assay. NM1NTZ exerted no adverse effects to cell viability at 400 μM of drug or less. FIG. 1D provides graphs showing the particle size, polydispersity index (PDI) and charge of the nanoformulations over time, thereby showing the stability of the nanoparticles.

FIGS. 2A-2D show drug uptake, retention, and cell viability. FIG. 2A provides a transmission electron microscopy (TEM) image of intracellular accumulation of a nanoformulation of a tenofovir alafenamide prodrug (NM1TAF; WO 2019/140365) after 8 hours of drug treatment. FIG. 2B shows the uptake of equal drug concentrations (10 μM) of NM1TAF and tenofovir alafenamide (TAF) by monocyte-derived macrophage (MDM) as determined by amount of prodrug (top) or active metabolite level (bottom). Uptake of NM1TAF was at least 10 times more than TAF control. FIG. 2C shows the retention of equal drug concentrations (10 μM) of NM1TAF and TAF by monocyte-derived macrophage (MDM) as determined by amount of prodrug (top) or active metabolite level (bottom). NM1TAF was retained in MDM to higher levels than TAF. FIG. 2D provides a graph of cell viability as evaluated by mitochondrial function in MDM by an MTT assay. NM1TAF exerted no adverse effects to cell viability at 200 μM of drug or less.

FIG. 3 provides graphs of hepatitis B virus (HBV) DNA after treatment (top) and human albumin levels before and after treatment (bottom). TK-NOG mice with a humanized liver were infected with 10⁶ genome equivalents (GE) of HBV. Two months after infection, a single intramuscular injection of the combination therapy of NM1TAF and NM1NTZ was administered at 75 mg/kg of native drug equivalents for each prodrug formulation. Reduction of HBV DNA in two animals below limit of detection (LOD) was found. These animals were euthanized to measure liver drug concentrations. * p values obtained by t-test statistically significant.

### DETAILED DESCRIPTION OF THE INVENTION

Herein, the preparation and characterization of long-acting prodrugs of thiazolide based drugs, particularly tizoxanide (TZ), are provided. Thiazolides (e.g., synthetic nitrothiazolyl-salicylamide derivatives or 2-hydroxyaroyl-N-(thiazol-2-yl)-amides) are a class of broad-spectrum antiviral drugs (Rossignol, J. F., Expert Opin. Drug Metab. Toxicol. (2009) 5(6):667-74; Rossignol, et al., Future Microbiol. (2008) 3(5):539-45; Keeffe, et al., World J. Gastroenterol. (2009) 15(15):1805-8; Rossignol, J. F., Antiviral Res. (2014) 110:94-103; Korba, et al., Antiviral Res. (2008) 77(1):56-63; La Frazia, et al., J. Virol. (2013) 87(20):11096-106). The development was initiated by creating modified TZ prodrugs (MTZ) then packaging them into nanoformulations (NMTZ) to improve drug biodistribution and plasma half-life. In a particular embodiment, the prodrugs comprise the native drug linked to a hydrophobic moiety (e.g., a fatty acid, alkyl or aryl moiety) via a cleavable moiety, particularly a hydrophobic moiety linked through a cleavable ester bond. Ester bond linkages are susceptible to enzymatic or chemical cleavage. In a particular embodiment, the nanoformulations comprise hydrophobic prodrug particles dispersed in an aqueous solution of polymeric excipients, lipids, or surfactants. Without being bound by theory, the mechanism of drug release involves dissolution of the prodrug from the excipient/nanoparticle followed by enzymatic or chemical hydrolysis of the prodrug to form the active agent.

Due to the improved drug biodistribution and bioavailability, improved cellular uptake and cellular retention (e.g., by monocyte-derived macrophages (MDM)), improved antiretroviral activity, and improved plasma half-life, the prodrugs and/or nanoformulations of the instant invention can be administered less frequently than native drug (e.g., once/month or longer). The prodrugs and/or nanoformulations of the instant invention can also be used in combination with long acting slow effective release (LASER) antiretroviral therapy (ART) such as ProTide LASER ART, particularly derivatives of nucleoside analogs conjugated to monophosphates masked with hydrophobic and lipophilic cleavable moieties (such as those described in WO 2019/140365 (incorporated by reference herein), particularly a tenofovir prodrug). The prodrugs and/or nanoformulations of the present invention can be used to treat, inhibit, and/or prevent diseases or disorders (e.g., diseases or disorders treated with the native thiazolide prodrug) including, without limitation: microbial infections (e.g., viral infections, bacterial infections, and/or parasitic infections (e.g., protozoa and/or helminths)), cancer, pain, neurodegenerative diseases, and aging-related disease. In a particular embodiment, the prodrugs and/or nanoformulations of the instant invention can be used to treat, inhibit, and/or prevent microbial infections such as viral infections, particularly human immunodeficiency virus (HIV, e.g., HIV-1), coronavirus (e.g., severe acute respiratory syndrome coronavirus 2 (SARS-CoV-2; COVID 19)) and hepatitis virus (e.g., hepatitis B virus (HBV; e.g., a chronic HBV infection)). Indeed, a combination of MTZ and LASER ProTide nanoformulations demonstrated sustained anti-HBV activity in humanized mice. The prodrugs and/or nanoformulations of the instant invention will improve patient compliance, affect drug targeting to reservoirs of infection, and reduce toxicities inherent in drug administration over prolonged time periods.

In a particular embodiment, the prodrugs of the instant invention are prodrugs of a thiazolide. Examples of thiazolides include, without limitation: tizoxanide; nitazoxanide; haloxanide (2-(hydroxyl)-N-(5-chloro-2-thiazolyl)benzamide); thiazolides described in Gargala, et al., Antimicrob. Agents Chemother. (2010) 54(3):1315-1318 (incorporated by reference herein), particularly those in Table 1 such as

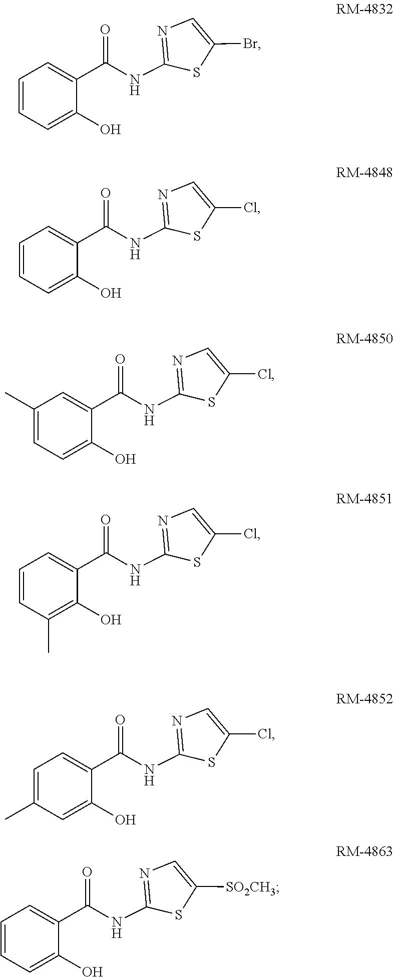 [SMILES: O=C(Nc1ncc(Br)s1)c1ccccc1O ; O=C(Nc1ncc(Cl)s1)c1ccccc1O ; Cc1ccc(O)c(C(=O)Nc2ncc(Cl)s2)c1 ; Cc1cccc(C(=O)Nc2ncc(Cl)s2)c1O ; Cc1ccc(C(=O)Nc2ncc(Cl)s2)c(O)c1 ; CS(=O)(=O)c1cnc(NC(=O)c2ccccc2O)s1]

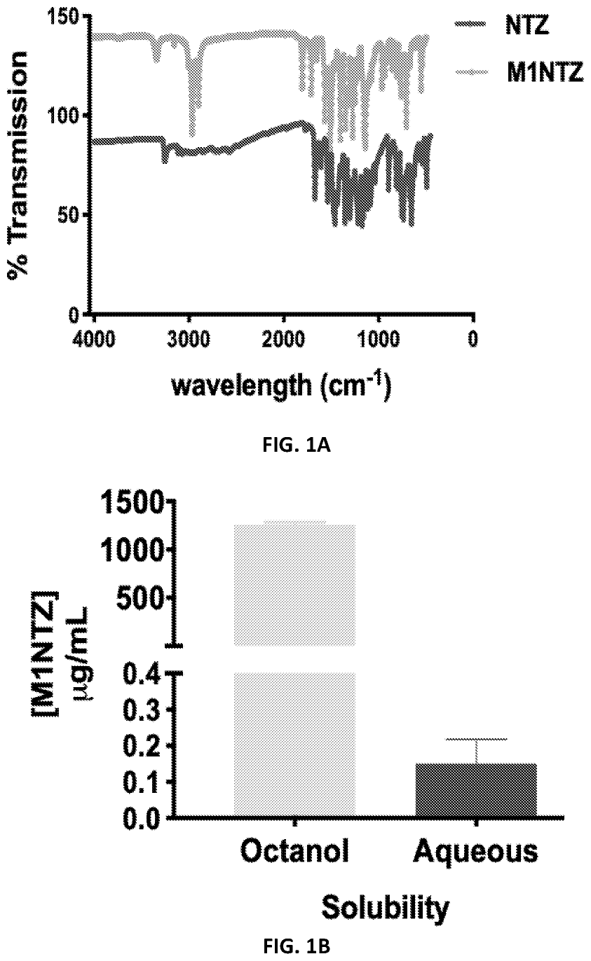

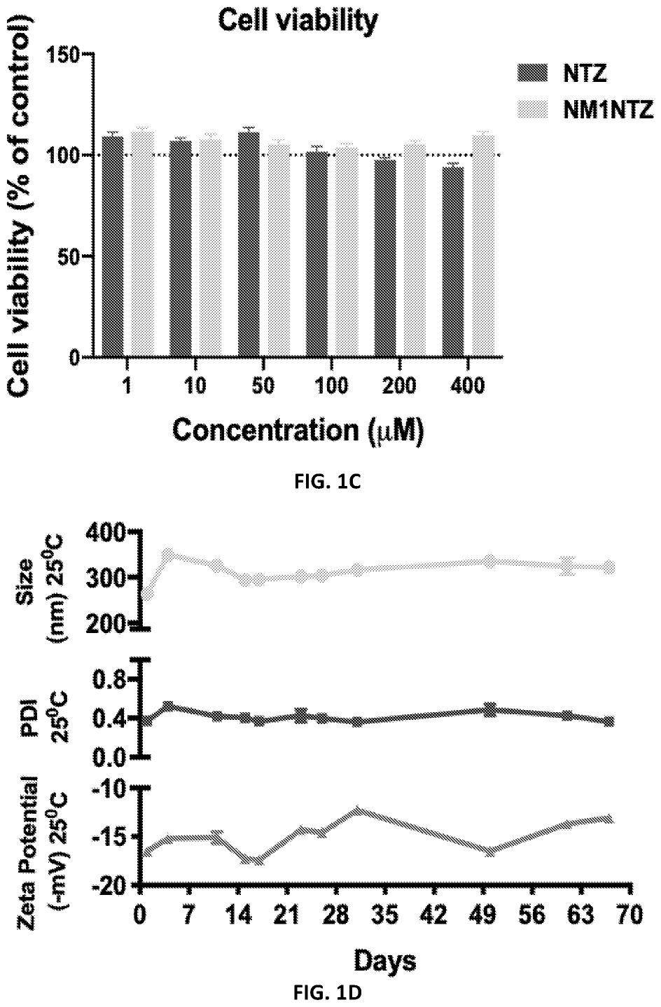

and thiazolides described in Stachulski et al., Future Med. Chem. (2018) 10: 851-862 (incorporated by reference herein), particularly those in Table 1 or FIG. 1.

The thiazolide compounds may be modified with a variety of side (e.g., hydrophobic) groups to generate prodrugs including but not limited to saturated, unsaturated, or branched aliphatic chains. The aliphatic chains may be substituted by heteroatoms such as O, N, or S. The side (e.g., hydrophobic) groups may also comprise aromatic moieties that can be substituted with heteroatoms such as O, N, or S. The side (e.g., hydrophobic) groups may also comprise an amino acid such as, without limitation: proline, alanine, or phenylalanine. In a particular embodiment, the side (e.g., hydrophobic) group comprises or consists of a saturated, unsaturated, or branched aliphatic chain that is between 4 and 24 carbon atoms. In a particular embodiment, two thiazolide compounds are linked by one of the side (e.g., hydrophobic) groups (e.g., thereby creating a dimer). The side group may contain an ester bond/linkage (e.g., the side (e.g., hydrophobic) group is attached to the native thiazolide compound (e.g., in place of an —OH or —OAc group (e.g., on the benzene ring). The ester bond/linkage may be cleavable.

In a particular embodiment, the prodrugs of the instant invention are derivatives of a thiazolide. In certain embodiments, a chemical moiety of the thiazolide, particularly an oxygen containing moiety such as a hydroxyl group or acetoxy group, has been replaced with an ester moiety (e.g., an ester moiety comprising a hydrophobic and/or lipophilic cleavable moiety). Prodrugs of the instant invention include, but are not limited to: fatty diester and monoester prodrugs, dimer prodrugs, and amino acid fatty esters.

In some embodiments, the prodrug of the present invention is a dimer of two thiazolides that are connected by a linker. The thiazolides in the dimer prodrug may be the same thiazolide or they may be different thiazolides. In a particular embodiment, the prodrug comprises a thiazolide wherein a chemical moiety, particularly an oxygen containing moiety such as a hydroxyl group or acetoxy group, is replaced with an ester comprising the linker. In a particular embodiment, the linker is an optionally substituted aliphatic or alkyl group. The aliphatic or alkyl group may be unsaturated or saturated, and may be substituted with at least one heteroatom (e.g., O, N, or S). In a particular embodiment, the alkyl or aliphatic group is hydrophobic. In a particular embodiment, the linker is an optionally substituted hydrocarbon chain, particularly saturated. In a particular embodiment, the linker a hydrocarbon chain. In a particular embodiment, the linker is a saturated linear aliphatic chain. In a particular embodiment, the alkyl or aliphatic group comprises about 1 to about 30 carbons (e.g., in the main chain of the alkyl or aliphatic group), which may be substituted with at least one heteroatom (e.g., O, N, or S). In a particular embodiment, the linker is about 1 to about 30 carbon atoms in length, 1 to about 28 carbons in length, 1 to about 26 carbons in length, 1 to about 24 carbons in length, 1 to about 22 carbons in length, 1 to about 20 carbons in length, 1 to about 18 carbons in length, 1 to about 16 carbons in length, 1 to about 10 carbons in length, about 10 to about 22 carbons in length, about 10 to about 20 carbons in length, about 12 to about 20 carbons in length, about 14 to about 18 carbons in length, about 14 to about 18 carbons in length, about 14 to about 20 carbons in length, about 15 to about 19 carbons in length, about 16 carbons in length, or about 17 carbons in length. Numbering here excludes the carbon in the C═O of the ester.

In some embodiments, the prodrug of the present invention is an amino acid fatty ester. In a particular embodiment, the prodrug comprises a thiazolide wherein a chemical moiety, particularly an oxygen containing moiety such as a hydroxyl group or a acetoxy group, is replaced with an amino acid fatty ester. The amino acid fatty ester may contain one or more amino acids, residues or side chains. In a particular embodiment, the amino fatty ester comprises 1 to 10 amino acids, particularly 1 to 7 amino acids, 1 to 5 amino acids, 1 to 4 amino acids, 1 to 3 amino acids, 1 to 2 amino acids, or 1 amino acid. In a particular embodiment, the amino fatty ester comprises only one amino acid, residue, or side chain. In a particular embodiment, the amino acid forms an amide bond with the C═O of the ester. In a particular embodiment, the prodrug comprises a thiazolide wherein an oxygen containing moiety such as a hydroxyl group or acetoxy group is replaced with the O of the amino acid carboxyl (—COOH) group. Any amino acid may be used. The amino acids of the amino acid fatty ester may be the same or different. In a particular embodiment, the amino acid is not charged (e.g., not aspartic acid, glutamic acid, arginine, lysine, or histidine). In a particular embodiment, the amino acid is hydrophobic. In a particular embodiment, the amino acid is selected from the group consisting of glycine, alanine, valine, leucine, isoleucine, proline, phenylalanine, methionine, and tryptophan. In a particular embodiment, the amino acid is selected from the group consisting of alanine, valine, phenylalanine, proline, tyrosine, and lysine. In a particular embodiment, the amino acid is selected from the group consisting of alanine, phenylalanine, and proline. In a particular embodiment, the amino acid is proline. In a particular embodiment, the amino acid fatty ester comprises a hydrophobic and/or lipophilic cleavable moiety (e.g., therapeutic fatty alcohols). In a particular embodiment, the hydrophobic and/or lipophilic cleavable moiety is the R group as defined hereinbelow.

In a particular embodiment, the prodrug of the instant invention is selected from the following group or a pharmaceutically acceptable salt or stereoisomer thereof:

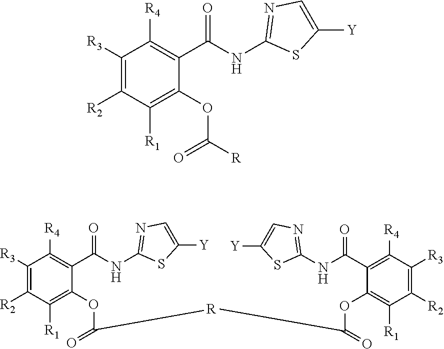

wherein R is a hydrophobic and/or lipophilic moiety; wherein R₁-R₄ are independently selected from the group consisting of hydrogen, hydroxyl, alkoxy, alkyl, and halogen; and wherein Y is selected from the group consisting of hydrogen, nitro, sulfonyl (e.g., methane sulfonyl), hydroxyl, alkoxy, alkyl, and halogen. In a particular embodiment, the carbon of the thiazole group adjacent to the carbon with the Y substituent may also be substituted with a substituent selected from the group consisting of hydrogen, nitro, methane sulfonyl, hydroxyl, alkoxy, alkyl, and halogen, particularly methyl or hydroxyl.

In a particular embodiment, at least two or three of R₁-R₄ are hydrogen. In a particular embodiment, R₁-R₄ are hydrogen. In a particular embodiment, when any of R₁-R₄ are not hydrogen, they are selected from the group consisting of hydroxyl, C₁-C₃ alkoxy, C₁-C₃ alkyl, and halogen. In a particular embodiment, when any of R₁-R₄ are not hydrogen, they are selected from the group consisting of hydroxyl, C₁-C₃ alkoxy, and C₁-C₃ alkyl. In a particular embodiment, when any of R₁-R₄ are not hydrogen, they are selected from the group consisting of hydroxyl, —OCH₃, and —CH₃.

In a particular embodiment, Y is selected from the group consisting of hydrogen, nitro, —CN, —SO₂CH₃, —SO₂CH₂CH₃, hydroxyl, C₁-C₃ alkoxy, C₁-C₃ alkyl, and halogen. In a particular embodiment, Y is selected from the group consisting of hydrogen, nitro, —CN, —SO₂CH₃, —SO₂CH₂CH₃, hydroxyl, —OCH₃, —SCH₃, —CH₃, CF₃, and halogen.

In a particular embodiment, R is a saturated or unsaturated linear or branched aliphatic chain, particularly in the range of 4 to 24 carbon atoms. The aliphatic chains may be substituted by heteroatoms such as O, N, or S. In a particular embodiment, R comprises an aromatic moiety that may be substituted with one or more heteroatom (e.g., N). In a particular embodiment, R comprises one or more amino acids (e.g., proline, alanine, or phenylalanine).

In a particular embodiment, R is the side chain of a fatty acid. The aliphatic or alkyl group may be unsaturated or saturated, and may be substituted with at least one heteroatom (e.g., O, N, or S). In a particular embodiment, R may contain an aromatic moiety that may be substituted with at least one heteroatom (e.g., O, N, or S). In a particular embodiment, R has between 1 and 24 carbons. In a particular embodiment, R has between 10 and 24 carbons.

In a particular embodiment, R is an alkyl or aliphatic group that is hydrophobic. In a particular embodiment, R is an optionally substituted hydrocarbon chain, particularly saturated. In a particular embodiment, R is a saturated linear aliphatic chain. In a particular embodiment, the alkyl or aliphatic group comprises about 1 to about 30 carbons, about 1 to about 24 carbons, or about 10 to about 24 carbons (e.g., in the main chain of the alkyl or aliphatic group), which may be substituted with at least one heteroatom (e.g., O, N, or S). In a particular embodiment, R is a C1-C29 unsaturated or saturated alkyl or aliphatic group, which may be substituted with at least one heteroatom (e.g., O, N, or S). In a particular embodiment, R is a C1-C24 unsaturated or saturated alkyl or aliphatic group, which may be substituted with at least one heteroatom (e.g., O, N, or S). In a particular embodiment, R is a C1-C21 unsaturated or saturated alkyl or aliphatic group, which may be substituted with at least one heteroatom (e.g., O, N, or S). In a particular embodiment, R is a C9-C29 unsaturated or saturated alkyl or aliphatic group, which may be substituted with at least one heteroatom (e.g., O, N, or S). In a particular embodiment, R is a C9-C21 unsaturated or saturated alkyl or aliphatic group, which may be substituted with at least one heteroatom (e.g., O, N, or S). In a particular embodiment, R is a C7-C23 unsaturated or saturated alkyl or aliphatic group, which may be substituted with at least one heteroatom (e.g., O, N, or S). In a particular embodiment, R is a C9-C21 unsaturated or saturated alkyl or aliphatic group, which may be substituted with at least one heteroatom (e.g., O, N, or S). In a particular embodiment, R is a C11-C19 unsaturated or saturated alkyl or aliphatic group, which may be substituted with at least one heteroatom (e.g., O, N, or S). In a particular embodiment, R is a C13-C19 unsaturated or saturated alkyl or aliphatic group, which may be substituted with at least one heteroatom (e.g., O, N, or S). In a particular embodiment, R is a C13-C17 unsaturated or saturated alkyl or aliphatic group, which may be substituted with at least one heteroatom (e.g., O, N, or S). In a particular embodiment, R is a C17 unsaturated or saturated alkyl or aliphatic group, which may be substituted with at least one heteroatom (e.g., O, N, or S). In a particular embodiment, R is a C15 unsaturated or saturated alkyl or aliphatic group, which may be substituted with at least one heteroatom (e.g., O, N, or S).

In a particular embodiment, R is the alkyl chain of a fatty acid (saturated or unsaturated), particularly a C4-C30 fatty acid, C6-C28 fatty acid, C8-C26 fatty acid a C10-C24 fatty acid, a C12-C22 fatty acid, a C14-C22 fatty acid, a C14-C20 fatty acid, a C14-C18 fatty acid, a C16-C18 fatty acid, a C18 fatty acid, or a C16 fatty acid (numbering here is inclusive of the carbon in the C═O of the ester).

In a particular embodiment, R is a saturated linear aliphatic chain or a hydrocarbon chain of at least 9 carbons (e.g., 9 to 24 carbons in length in the chain, 9 to 21 carbons in length in the chain, 9 to 19 carbons in length in the chain, 11 to 17 carbons in length in the chain, 13 to 21 carbons in length in the chain, 13 to 19 carbons in length in the chain, 15 to 19 carbons in length in the chain, or 15 or 17 carbons in length in the chain). In a particular embodiment, R is a saturated linear aliphatic chain or a hydrocarbon chain of 12, 13, 14, 15, 16, 17, 18, 19, 20, or 21 carbons in length, particularly 12, 13, 14, 15, 16, 17, 18, or 19 carbons in length, 15, 16, 17, 18, or 19 carbons in length, or 17 carbons in length. In a particular embodiment, R is a saturated linear aliphatic chain or a hydrocarbon chain of 17 carbons in length.

In a particular embodiment, the prodrug of the instant invention is selected from the following group or a pharmaceutically acceptable salt or stereoisomer thereof:

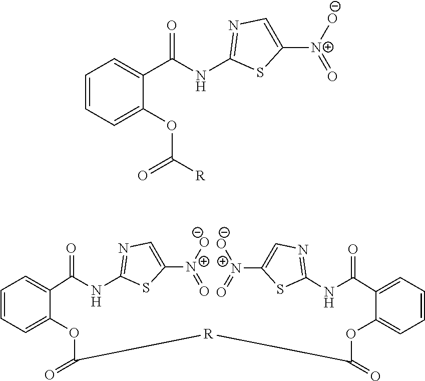

wherein R is a hydrophobic and/or lipophilic moiety.

In a particular embodiment, R is a saturated or unsaturated linear or branched aliphatic chain, particularly in the range of 4 to 24 carbon atoms. The aliphatic chains may be substituted by heteroatoms such as O, N, or S. In a particular embodiment, R comprises an aromatic moiety that may be substituted with one or more heteroatom (e.g., N). In a particular embodiment, R comprises one or more amino acids (e.g., proline, alanine, or phenylalanine).

In a particular embodiment, R is the side chain of a fatty acid. The aliphatic or alkyl group may be unsaturated or saturated, and may be substituted with at least one heteroatom (e.g., O, N, or S). In a particular embodiment, R may contain an aromatic moiety that may be substituted with at least one heteroatom (e.g., O, N, or S). In a particular embodiment, R has between 1 and 24 carbons. In a particular embodiment, R has between 10 and 24 carbons.

In a particular embodiment, R is an alkyl or aliphatic group that is hydrophobic. In a particular embodiment, R is an optionally substituted hydrocarbon chain, particularly saturated. In a particular embodiment, R is a saturated linear aliphatic chain. In a particular embodiment, the alkyl or aliphatic group comprises about 1 to about 30 carbons, about 1 to about 24 carbons, or about 10 to about 24 carbons (e.g., in the main chain of the alkyl or aliphatic group), which may be substituted with at least one heteroatom (e.g., O, N, or S). In a particular embodiment, R is a C1-C29 unsaturated or saturated alkyl or aliphatic group, which may be substituted with at least one heteroatom (e.g., O, N, or S). In a particular embodiment, R is a C1-C24 unsaturated or saturated alkyl or aliphatic group, which may be substituted with at least one heteroatom (e.g., O, N, or S). In a particular embodiment, R is a C1-C21 unsaturated or saturated alkyl or aliphatic group, which may be substituted with at least one heteroatom (e.g., O, N, or S). In a particular embodiment, R is a C9-C29 unsaturated or saturated alkyl or aliphatic group, which may be substituted with at least one heteroatom (e.g., O, N, or S). In a particular embodiment, R is a C9-C21 unsaturated or saturated alkyl or aliphatic group, which may be substituted with at least one heteroatom (e.g., O, N, or S). In a particular embodiment, R is a C7-C23 unsaturated or saturated alkyl or aliphatic group, which may be substituted with at least one heteroatom (e.g., O, N, or S). In a particular embodiment, R is a C9-C21 unsaturated or saturated alkyl or aliphatic group, which may be substituted with at least one heteroatom (e.g., O, N, or S). In a particular embodiment, R is a C11-C19 unsaturated or saturated alkyl or aliphatic group, which may be substituted with at least one heteroatom (e.g., O, N, or S). In a particular embodiment, R is a C13-C19 unsaturated or saturated alkyl or aliphatic group, which may be substituted with at least one heteroatom (e.g., O, N, or S). In a particular embodiment, R is a C13-C17 unsaturated or saturated alkyl or aliphatic group, which may be substituted with at least one heteroatom (e.g., O, N, or S). In a particular embodiment, R is a C17 unsaturated or saturated alkyl or aliphatic group, which may be substituted with at least one heteroatom (e.g., O, N, or S). In a particular embodiment, R is a C15 unsaturated or saturated alkyl or aliphatic group, which may be substituted with at least one heteroatom (e.g., O, N, or S).

In a particular embodiment, R is the alkyl chain of a fatty acid (saturated or unsaturated), particularly a C4-C30 fatty acid, C6-C28 fatty acid, C8-C26 fatty acid a C10-C24 fatty acid, a C12-C22 fatty acid, a C14-C22 fatty acid, a C14-C20 fatty acid, a C14-C18 fatty acid, a C16-C18 fatty acid, a C18 fatty acid, or a C16 fatty acid (numbering here is inclusive of the carbon in the C═O of the ester).

In a particular embodiment, R is a saturated linear aliphatic chain or a hydrocarbon chain of at least 9 carbons (e.g., 9 to 24 carbons in length in the chain, 9 to 21 carbons in length in the chain, 9 to 19 carbons in length in the chain, 11 to 17 carbons in length in the chain, 13 to 21 carbons in length in the chain, 13 to 19 carbons in length in the chain, 15 to 19 carbons in length in the chain, or 15 or 17 carbons in length in the chain). In a particular embodiment, R is a saturated linear aliphatic chain or a hydrocarbon chain of 12, 13, 14, 15, 16, 17, 18, 19, 20, or 21 carbons in length, particularly 12, 13, 14, 15, 16, 17, 18, or 19 carbons in length, 15, 16, 17, 18, or 19 carbons in length, or 17 carbons in length. In a particular embodiment, R is a saturated linear aliphatic chain or a hydrocarbon chain of 17 carbons in length.

In a particular embodiment, the prodrug of the instant invention is:

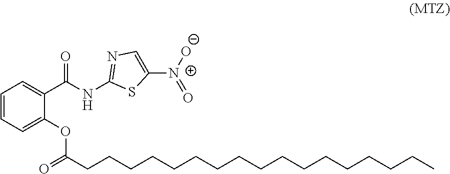 [SMILES: CCCCCCCCCCCCCCCCCC(=O)Oc1ccccc1C(=O)Nc1ncc([N+](=O)[O-])s1]

or a pharmaceutically acceptable salt or stereoisomer thereof.

The instant invention also encompasses nanoparticles (sometimes referred to herein as nanoformulations) comprising the prodrug of the instant invention. The nanoparticles may be used for the delivery of the compounds to a cell or host (e.g., in vitro or in vivo). In a particular embodiment, the nanoparticle is used for the delivery of antiretroviral therapy to a subject. The nanoparticles of the instant invention comprise at least one prodrug and at least one surfactant or polymer. In a particular embodiment, the nanoparticles comprise a spectroscopic-defined surfactant/polymer:drug ratio that maintains optimal targeting of the drug nanoparticle to maintain a macrophage depot. These components of the nanoparticle, along with other optional components, are described hereinbelow.

Methods of synthesizing the nanoparticles of the instant invention are known in the art. In a particular embodiment, the methods generate nanoparticles comprising a prodrug (e.g., crystalline or amorphous) coated (either partially or completely) with a polymer and/or surfactant. Examples of synthesis methods include, without limitation, milling (e.g., wet milling), homogenization (e.g., high pressure homogenization), particle replication in nonwetting template (PRINT) technology, and/or sonication techniques. For example, U.S. Patent Application Publication No. 2013/0236553, incorporated by reference herein, provides methods suitable for synthesizing nanoparticles of the instant invention. In a particular embodiment, the polymers or surfactants are firstly chemically modified with targeting ligands and then used directly or mixed with non-targeted polymers or surfactants in certain molar ratios to coat on the surface of prodrug suspensions—e.g., by using a nanoparticle synthesis process (e.g., a crystalline nanoparticle synthesis process) such as milling (e.g., wet milling), homogenization (e.g., high pressure homogenization), particle replication in nonwetting template (PRINT) technology, and/or sonication techniques, thereby preparing targeted nanoformulations. The nanoparticles may be used with or without further purification, although the avoidance of further purification is desirable for quicker production of the nanoparticles. In a particular embodiment, the nanoparticles are synthesized using milling and/or homogenization. Targeted nanoparticles (e.g., using ligands (optionally with high molecular weight)) may be developed through either physically or chemically coating and/or binding on the surface of polymers or surfactants and/or prodrug nanosuspensions.

In a particular embodiment, the nanoparticles of the instant invention are synthesized by adding the prodrug (e.g., crystals) to a polymer or surfactant solution and then generating the nanoparticles (e.g., by wet milling or high pressure homogenization). The prodrug and polymer or surfactant solution may be agitated prior to the wet milling or high pressure homogenization.

The nanoparticles of the instant invention may be used to deliver at least one prodrug of the instant invention to a cell or a subject (including non-human animals). In a particular embodiment, the nanoparticle comprises more than one unique prodrug of the instant invention. The nanoparticles of the instant invention may further comprise at least one other agent or compound, particularly a bioactive agent, particularly a therapeutic agent (e.g., antiviral compound) or diagnostic agent, particularly at least one antiviral or antiretroviral. In a particular embodiment, the nanoparticles of the instant invention comprise at least two therapeutic agents, particularly wherein at least one is a prodrug of the instant invention. For example, the nanoparticle may comprise a prodrug of the instant invention and at least one other therapeutic agent (e.g., an anti-HIV agent, and anti-HBV agent, anti-coronavirus agent).

In a particular embodiment, the nanoparticles of the instant invention are a submicron colloidal dispersion of nanosized drug/prodrug crystals stabilized by polymers or surfactants (e.g., surfactant-coated drug crystals; a nanoformulation). In a particular embodiment, the prodrug and/or nanoparticle is crystalline (solids having the characteristics of crystals), amorphous, or are solid-state nanoparticles of the prodrug that is formed as crystal that combines the prodrug and polymer or surfactant. In a particular embodiment, the prodrug of the nanoparticle is crystalline. As used herein, the term “crystalline” refers to an ordered state (i.e., non-amorphous) and/or a substance exhibiting long-range order in three dimensions. In a particular embodiment, the majority (e.g., at least 50%, 60%, 70%, 80%, 90%, 95% or more) of the prodrug and, optionally, the hydrophobic portion of the surfactant or polymer are crystalline.

In a particular embodiment, the nanoparticle of the instant invention is up to about 2 or 3 μm in diameter (e.g., z-average diameter) or its longest dimension, particularly up to about 1 μm (e.g., about 100 nm to about 1 μm). For example, the diameter or longest dimension of the nanoparticle may be about 50 to about 800 nm. In a particular embodiment, the diameter or longest dimension of the nanoparticle is about 50 to about 750 nm, about 50 to about 600 nm, about 50 to about 500 nm, about 200 nm to about 600 nm, about 200 nm to about 500 nm, about 200 nm to about 400 nm, about 250 nm to about 350 nm, or about 250 nm to about 400 nm. The nanoparticles may be, for example, rod shaped, elongated rods, irregular, or round shaped. The nanoparticles of the instant invention may be neutral or charged. The nanoparticles may be charged positively or negatively.

As stated hereinabove, the nanoparticles of the instant invention comprise at least one polymer or surfactant. A “surfactant” refers to a surface-active agent, including substances commonly referred to as wetting agents, detergents, dispersing agents, or emulsifying agents. Surfactants are usually organic compounds that are amphiphilic.

Examples of polymers or surfactants include, without limitation, synthetic or natural phospholipids, PEGylated lipids (e.g., PEGylated phospholipid), lipid derivatives, polysorbates, amphiphilic copolymers, amphiphilic block copolymers, poly(ethylene glycol)-co-poly(lactide-co-glycolide) (PEG-PLGA), their derivatives, ligand-conjugated derivatives and combinations thereof. Other polymers or surfactants and their combinations that can form stable nanosuspensions and/or can chemically/physically bind to the targeting ligands of HIV infectable/infected CD4+ T cells, macrophages and dendritic cells can be used in the instant invention. Further examples of polymers or surfactants include, without limitation: 1) nonionic surfactants (e.g., pegylated and/or polysaccharide-conjugated polyesters and other hydrophobic polymeric blocks such as poly(lactide-co-glycolide) (PLGA), polylactic acid (PLA), polycaprolactone (PCL), other polyesters, poly(propylene oxide), poly(1,2-butylene oxide), poly(n-butylene oxide), poly(tetrahydrofurane), and poly(styrene); glyceryl esters, polyoxyethylene fatty alcohol ethers, polyoxyethylene sorbitan fatty acid esters, polyoxyethylene fatty acid esters, sorbitan esters, glycerol monostearate, polyethylene glycols, polypropyleneglycols, cetyl alcohol, cetostearyl alcohol, stearyl alcohol, aryl alkyl polyether alcohols, polyoxyethylene-polyoxypropylene copolymers, poloxamines, cellulose, methylcellulose, hydroxylmethylcellulose, hydroxypropylcellulose, hydroxypropylmethylcellulose, polysaccharides, starch and their derivatives, hydroxyethylstarch, polyvinyl alcohol (PVA), polyvinylpyrrolidone, and their combination thereof); and 2) ionic surfactants (e.g., phospholipids, amphiphilic lipids, 1,2-dialkylglycero-3-alkylphophocholines, 1, 2-distearoyl-sn-glecro-3-phosphocholine (DSPC), 1,2-distearoyl-sn-glycero-3-phosphoethanolamine-N-[carboxy(polyethylene glycol) (DSPE-PEG), dimethylaminoethanecarbamoyl cheolesterol (DC-Chol), N-[1-(2,3-Dioleoyloxy)propyl]-N,N,N-trimethylammonium (DOTAP), alkyl pyridinium halides, quaternary ammonium compounds, lauryldimethylbenzylammonium, acyl carnitine hydrochlorides, dimethyldioctadecylammonium (DDAB), n-octylamines, oleylamines, benzalkonium, cetyltrimethylammonium, chitosan, chitosan salts, poly(ethylenimine) (PEI), poly(N-isopropyl acrylamide (PNIPAM), and poly(allylamine) (PAH), poly (dimethyldiallylammonium chloride) (PDDA), alkyl sulfonates, alkyl phosphates, alkyl phosphonates, potassium laurate, triethanolamine stearate, sodium lauryl sulfate, sodium dodecylsulfate, alkyl polyoxyethylene sulfates, alginic acid, alginic acid salts, hyaluronic acid, hyaluronic acid salts, gelatins, dioctyl sodium sulfosuccinate, sodium carboxymethylcellulose, cellulose sulfate, dextran sulfate and carboxymethylcellulose, chondroitin sulfate, heparin, synthetic poly(acrylic acid) (PAA), poly (methacrylic acid) (PMA), poly(vinyl sulfate) (PVS), poly(styrene sulfonate) (PSS), bile acids and their salts, cholic acid, deoxycholic acid, glycocholic acid, taurocholic acid, glycodeoxycholic acid, derivatives thereof, and combinations thereof.

The polymer or surfactant of the instant invention may be charged or neutral. In a particular embodiment, the polymer or surfactant is neutral or negatively charged (e.g., poloxamers, polysorbates, phospholipids, and their derivatives).

In a particular embodiment, the polymer or surfactant is an amphiphilic block copolymer or lipid derivative. In a particular embodiment, at least one polymer or surfactant of the nanoparticle is an amphiphilic block copolymer, particularly a copolymer comprising at least one block of poly(oxyethylene) and at least one block of poly(oxypropylene). In a particular embodiment, the polymer or surfactant is a triblock amphiphilic block copolymer. In a particular embodiment, the polymer or surfactant is a triblock amphiphilic block copolymer comprising a central hydrophobic block of polypropylene glycol flanked by two hydrophilic blocks of polyethylene glycol. In a particular embodiment, the surfactant is poloxamer 407.

In a particular embodiment, the amphiphilic block copolymer is a copolymer comprising at least one block of poly(oxyethylene) and at least one block of poly(oxypropylene). In a particular embodiment, the amphiphilic block copolymer is a poloxamer. Examples of poloxamers include, without limitation, Pluronic® L31, L35, F38, L42, L43, L44, L61, L62, L63, L64, P65, F68, L72, P75, F77, L81, P84, P85, F87, F88, L92, F98, L101, P103, P104, P105, F108, L121, L122, L123, F127, 10R5, 10R8, 12R3, 17R1, 17R2, 17R4, 17R8, 22R4, 25R1, 25R2, 25R4, 25R5, 25R8, 31R1, 31R2, and 31R4. In a particular embodiment, the poloxamer is poloxamer 407 (Pluronic® F127).

In a particular embodiment of the invention, the polymer or surfactant is present in the nanoparticle and/or solution to synthesize the nanoparticle (as described herein) at a concentration ranging from about 0.0001% to about 10% or 15% by weight. In a particular embodiment, the concentration of the polymer or surfactant ranges from about 0.01% to about 15%, about 0.01% to about 10%, about 0.1% to about 10%, or about 0.1% to about 6% by weight. In a particular embodiment, the nanoparticle comprises at least about 50%, 75%, 80%, 85%, 90%, 95%, 97%, 98%, 99% or higher therapeutic agent (prodrug) by weight. In a particular embodiment, the nanoparticles comprise a defined drug:polymer/surfactant ratio. In a particular embodiment, the drug:polymer/surfactant ratio (e.g., by weight) is from about 1:1 to about 1000:1, about 1:1 to about 10:1, about 10:6 to about 1000:6, about 20:6 to about 500:6, about 50:6 to about 200:6, or about 100:6.

As stated hereinabove, the polymer or surfactant of the instant invention may be linked to a targeting ligand. The targeting of the nanoparticles (e.g., to macrophage) can provide for superior targeting, decreased excretion rates, decreased toxicity, and prolonged half-life compared to free drug or non-targeted nanoparticles. A targeting ligand is a compound that specifically binds to a specific type of tissue or cell type (e.g., in a desired target:cell ratio). For example, a targeting ligand may be used for engagement or binding of a target cell (e.g., a macrophage, T cell, dendritic cell, etc.) surface marker or receptor which may facilitate its uptake into the cell (e.g., within a protected subcellular organelle that is free from metabolic degradation). In a particular embodiment, the targeting ligand is a ligand for a cell surface marker/receptor. The targeting ligand may be an antibody or antigen-binding fragment thereof immunologically specific for a cell surface marker (e.g., protein or carbohydrate) preferentially or exclusively expressed on the targeted tissue or cell type. The targeting ligand may be linked directly to the polymer or surfactant or via a linker. Generally, the linker is a chemical moiety comprising a covalent bond or a chain of atoms that covalently attaches the ligand to the polymer or surfactant. The linker can be linked to any synthetically feasible position of the ligand and the polymer or surfactant. Exemplary linkers may comprise at least one optionally substituted; saturated or unsaturated; linear, branched or cyclic aliphatic group, an alkyl group, or an optionally substituted aryl group. The linker may be a lower alkyl or aliphatic. The linker may also be a polypeptide (e.g., from about 1 to about 10 amino acids, particularly about 1 to about 5). In a particular embodiment, the targeting moiety is linked to either or both ends of the polymer or surfactant. The linker may be non-degradable and may be a covalent bond or any other chemical structure which cannot be substantially cleaved or cleaved at all under physiological environments or conditions.

The nanoparticles/nanoformulations of the instant invention may comprise targeted and/or non-targeted polymers or surfactants. In a particular embodiment, the molar ratio of targeted and non-targeted polymers or surfactants in the nanoparticles/nanoformulations of the instant invention is from about 0.001 to 100%, about 1% to about 99%, about 5% to about 95%, about 10% to about 90%, about 25% to about 75%, about 30% to about 60%, or about 40%. In a particular embodiment, the nanoparticle comprises only targeted polymers or surfactants. In a particular embodiment, the nanoparticles/nanoformulations of the instant invention comprise a folate targeted polymer or surfactant and a non-targeted version of the polymer or surfactant. In a particular embodiment, the nanoparticles/nanoformulations of the instant invention comprise folate-poloxamer 407 (FA-P407) and/or poloxamer 407.

Examples of targeting ligands include but are not limited to macrophage targeting ligands, CD4+ T cell targeting ligands, dendritic cell targeting ligands, and tumor targeting ligands. In a particular embodiment, the targeting ligand is a macrophage targeting ligand. The targeted nanoformulations of the instant invention may comprise a targeting ligand for directing the nanoparticles to HIV tissue and cellular sanctuaries/reservoirs (e.g., central nervous system, gut associated lymphoid tissues (GALT), CD4+ T cells, macrophages, dendritic cells, etc.). Macrophage targeting ligands include, without limitation, folate receptor ligands (e.g., folate (folic acid) and folate receptor antibodies and fragments thereof (see, e.g., Sudimack et al. (2000) Adv. Drug Del. Rev., 41:147-162)), mannose receptor ligands (e.g., mannose), formyl peptide receptor (FPR) ligands (e.g., N-formyl-Met-Leu-Phe (fMLF) (SEQ ID NO: 1)), and tuftsin (the tetrapeptide Thr-Lys-Pro-Arg (SEQ ID NO: 2)). Other targeting ligands include, without limitation, hyaluronic acid, gp120 and peptide fragments thereof, and ligands or antibodies specific for CD4, CCR5, CXCR4, CD7, CD111, CD204, CD49a, CD29, CD19, CD20, CD22, CD171, CD33, Leis-Y, WT-1, ROR1, MUC16, MUC1, MUC4, estrogen receptor, transferrin receptors, EGF receptors (e.g. HER2), folate receptor, VEGF receptor, FGF receptor, androgen receptor, NGR, Integrins, and GD2. In a particular embodiment, the targeting ligand is folic acid.

As stated hereinabove, the nanoparticles of the instant invention may comprise a further therapeutic agent. The instant invention also encompasses therapeutic methods wherein the prodrug and/or nanoparticles of the instant invention are co-administered with another therapeutic agent (e.g., sequentially and/or simultaneously). In a particular embodiment, the therapeutic agent is hydrophobic, a water insoluble compound, or a poorly water soluble compound, particularly when included in the nanoparticle. For example, the therapeutic agent may have a solubility of less than about 10 mg/ml, less than 1 mg/ml, more particularly less than about 100 μg/ml, and more particularly less than about 25 μg/ml in water or aqueous media in a pH range of 0-14, preferably between pH 4 and 10, particularly at 20° C.

In a particular embodiment, the therapeutic agent is an antiviral or an antiretroviral. In a particular embodiment, the therapeutic agent is an anti-HBV agent or an anti-coronavirus agent. Examples of anti-HBV agents include without limitation tenofovir (e.g., tenofovir disoproxil, tenofovir alafenamide), entecavir, telbivudine, adefovir (e.g., adefovir dipivoxil), lamivudine, and immune modulators such as interferons (e.g., pegylated interferon) and interferon alpha.

The antiretroviral may be effective against or specific to lentiviruses. Lentiviruses include, without limitation, human immunodeficiency virus (HIV) (e.g., HIV-1, HIV-2), bovine immunodeficiency virus (BIV), feline immunodeficiency virus (FIV), simian immunodeficiency virus (SIV), and equine infectious anemia virus (EIA). In a particular embodiment, the therapeutic agent is an anti-HIV agent. An anti-HIV compound or an anti-HIV agent is a compound which inhibits HIV (e.g., inhibits HIV replication and/or infection). Examples of anti-HIV agents include, without limitation:

(I) Nucleoside-analog reverse transcriptase inhibitors (NRTIs). NRTIs refer to nucleosides and nucleotides and analogues thereof that inhibit the activity of reverse transcriptase, particularly HIV-1 reverse transcriptase. NRTIs comprise a sugar and base. Examples of nucleoside-analog reverse transcriptase inhibitors include, without limitation, adefovir dipivoxil, adefovir, lamivudine, telbivudine, entecavir, tenofovir, stavudine, abacavir, didanosine, emtricitabine, zalcitabine, and zidovudine.

(II) Non-nucleoside reverse transcriptase inhibitors (NNRTIs). NNRTIs are allosteric inhibitors which bind reversibly at a nonsubstrate-binding site on reverse transcriptase, particularly the HIV reverse transcriptase, thereby altering the shape of the active site or blocking polymerase activity. Examples of NNRTIs include, without limitation, delavirdine (DLV, BHAP, U-90152; Rescriptor®), efavirenz (EFV, DMP-266, SUSTIVA®), nevirapine (NVP, Viramune®), PNU-142721, capravirine (S-1153, AG-1549), emivirine (+)-calanolide A (NSC-675451) and B, etravirine (ETR, TMC-125, Intelence®), rilpivirne (RPV, TMC278, Edurant™) DAPY (TMC120), doravirine (Pifeltro™), BILR-355 BS, PHI-236, and PHI-443 (TMC-278).

(III) Protease inhibitors (PI). Protease inhibitors are inhibitors of a viral protease, particularly the HIV-1 protease. Examples of protease inhibitors include, without limitation, darunavir, amprenavir (141W94, AGENERASE®), tipranivir (PNU-140690, APTIVUS®), indinavir (MK-639; CRIXIVAN®), saquinavir (INVIRASE®, FORTOVASE®), fosamprenavir (LEXIVA®), lopinavir (ABT-378), ritonavir (ABT-538, NORVIR®), atazanavir (REYATAZ®), nelfinavir (AG-1343, VIRACEPT®), lasinavir (BMS-234475/CGP-61755), BMS-2322623, GW-640385X (VX-385), AG-001859, and SM-309515.

(IV) Fusion or entry inhibitors. Fusion or entry inhibitors are compounds, such as peptides, which block HIV entry into a cell (e.g., by binding to HIV envelope protein and blocking the structural changes necessary for the virus to fuse with the host cell). Examples of fusion inhibitors include, without limitation, CCR5 receptor antagonists (e.g., maraviroc (Selzentry®, Celsentri)), enfuvirtide (INN, FUZEON®), T-20 (DP-178, FUZEON®) and T-1249.

(V) Integrase inhibitors. Integrase inhibitors are a class of antiretroviral drug designed to block the action of integrase (e.g., HIV integrase), a viral enzyme that inserts the viral genome into the DNA of the host cell. Examples of integrase inhibitors include, without limitation, raltegravir, elvitegravir, GSK1265744 (cabotegravir), GSK1349572 (dolutegravir), GS-9883 (bictegravir), and MK-2048.

Anti-HIV compounds also include maturation inhibitors (e.g., bevirimat). Maturation inhibitors are typically compounds which bind HIV gag and disrupt its processing during the maturation of the virus. Anti-HIV compounds also include HIV vaccines such as, without limitation, ALVAC® HIV (vCP1521), AIDSVAX® B/E (gp120), and combinations thereof. Anti-HIV compounds also include HIV antibodies (e.g., antibodies against gp120 or gp41), particularly broadly neutralizing antibodies.

More than one anti-HIV agent may be used, particularly where the agents have different mechanisms of action (as outlined above). For example, anti-HIV agents which are not NNRTIs may be combined with the NNRTI prodrugs of the instant invention. In a particular embodiment, the anti-HIV therapy is highly active antiretroviral therapy (HAART).

In a particular embodiment, the prodrug and/or the nanoformulation of the prodrug is used in combination with a long acting slow effective release ART (LASER ART) formulations (such as described in WO 2020/112931, WO 2020/086555, WO 2019/199756, U.S. patent application Ser. No. 16/304,759, and U.S. Patent Application Publication No. 20170304308, each of the foregoing incorporated by reference herein) and/or ProTide LASER ART formulations (e.g., as described in WO 2019/140365, incorporated by reference herein). For example, the prodrug and/or the nanoformulation of the prodrug of the instant invention is administered with or formulated with (e.g., in the same composition or nanoparticle) with a long acting slow effective release ART (LASER ART) formulation and/or ProTide LASER ART formulation. In a particular embodiment, the combination is used to treat a viral infection including but not limited to HIV or hepatitis B. In a particular embodiment, the prodrug and/or the nanoformulation of the prodrug is used in combination with a long acting slow effective release ART (LASER ART) formulation and/or ProTide LASER ART formulation of tenofovir, particularly those provided in WO 2019/140365 (incorporated by reference herein). For example, the prodrug may have the formula

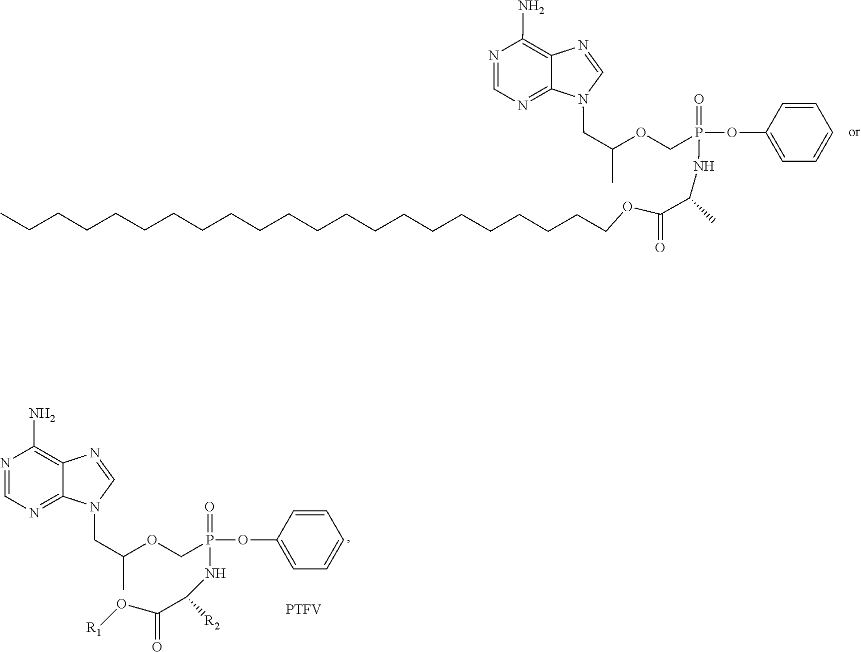

wherein R₁ is C₂₂ hydrocarbon and R₂ is methyl or benzyl.

The instant invention encompasses compositions (e.g., pharmaceutical compositions) comprising at least one prodrug and/or nanoparticle of the instant invention and at least one pharmaceutically acceptable carrier. As stated hereinabove, the nanoparticle may comprise more than one therapeutic agent. In a particular embodiment, the pharmaceutical composition comprises a first nanoparticle comprising a first prodrug and a second nanoparticle comprising a second prodrug, wherein the first and second prodrugs are different. In a particular embodiment, the first prodrug is a prodrug of the instant invention and the second prodrug is a prodrug of a non-nucleoside reverse transcriptase inhibitor (NNRTI), particularly rilpivirine (RPV). The compositions (e.g., pharmaceutical compositions) of the instant invention may further comprise other therapeutic agents (e.g., other anti-HIV compounds (e.g., those described herein)).

The present invention also encompasses methods for preventing, inhibiting, and/or treating a disease or disorder. The methods comprise administering a prodrug and/or nanoparticle of the instant invention (optionally in a composition) to a subject in need thereof. The prodrugs and/or nanoformulations of the present invention can be used for the treatment and/or prevention of diseases including but not limited to viral infections, bacterial infections, and parasitic infections, cancer, pain, neurodegenerative diseases, and aging-related diseases. Viral infections include, but are not limited to: Hepatitis A infections, Hepatitis B infections, Hepatitis C infections, HIV infections, Influenza infections, Rhinovirus infections, Adenovirus infections, Parainfluenza infections, Rotavirus infections, Norovirus infections, coronavirus infections, SARS infections, and respiratory syncytial virus infections. Parasitic infections include, but are not limited to: Giardia infections, Entamoeba infections, Cryptosporidium infections, cyclospora infections, Trichomonas infections, Encephalitozoon intestinalis infections, Isospora belli infections, Blasocystis hominis infections, Ascaris infections, Trichuris trichura infections, Taenia saginata infections, Hymenolepis nana infections, Fasciola hepatica infections, and Balantidium coli. infections. Bacterial infections include, but are not limited to: Bacteroides based infections, Clostridium based infections, Helicobacter pylori infections, and other aerobic and anaerobic gram positive and gram negative based bacterial infections. In a particular embodiment, the disease or disorder is a viral (e.g., retroviral) infection. Examples of viral infections include, without limitation: HIV, Hepatitis B, Hepatitis C, and HTLV. In a particular embodiment, the viral infection is a retroviral or lentiviral infection, particularly an HIV infection (e.g., HIV-1).

The prodrugs and/or nanoparticles of the instant invention (optionally in a composition) can be administered to an animal, in particular a mammal, more particularly a human, in order to treat/inhibit/prevent the disease or disorder (e.g., a retroviral infection such as an HIV infection). The pharmaceutical compositions of the instant invention may also comprise at least one other therapeutic agent such as an antiviral agent, particularly at least one other anti-HIV compound/agent. The additional anti-HIV compound may also be administered in a separate pharmaceutical composition from the prodrugs or compositions of the instant invention. The pharmaceutical compositions may be administered at the same time or at different times (e.g., sequentially).

The dosage ranges for the administration of the prodrugs, nanoparticles, and/or compositions of the invention are those large enough to produce the desired effect (e.g., curing, relieving, treating, and/or preventing the disease or disorder (e.g., HIV infection), the symptoms of it (e.g., AIDS, ARC), or the predisposition towards it). In a particular embodiment, the pharmaceutical composition of the instant invention is administered to the subject at an amount from about 5 μg/kg to about 500 mg/kg. In a particular embodiment, the pharmaceutical composition of the instant invention is administered to the subject at an amount greater than about 5 μg/kg, greater than about 50 μg/kg, greater than about 0.1 mg/kg, greater than about 0.5 mg/kg, greater than about 1 mg/kg, or greater than about 5 mg/kg. In a particular embodiment, the pharmaceutical composition of the instant invention is administered to the subject at an amount from about 0.5 mg/kg to about 100 mg/kg, about 10 mg/kg to about 100 mg/kg, or about 15 mg/kg to about 50 mg/kg. The dosage should not be so large as to cause significant adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications.

The prodrugs and nanoparticles described herein will generally be administered to a patient as a pharmaceutical composition. The term “patient” as used herein refers to human or animal subjects. These prodrugs and nanoparticles may be employed therapeutically, under the guidance of a physician.

The pharmaceutical compositions comprising the prodrugs and/or nanoparticles of the instant invention may be conveniently formulated for administration with any pharmaceutically acceptable carrier(s). For example, the complexes may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents, or suitable mixtures thereof, particularly an aqueous solution. The concentration of the prodrugs and/or nanoparticles in the chosen medium may be varied and the medium may be chosen based on the desired route of administration of the pharmaceutical composition. Except insofar as any conventional media or agent is incompatible with the nanoparticles to be administered, its use in the pharmaceutical composition is contemplated.

The dose and dosage regimen of prodrugs and/or nanoparticles according to the invention that are suitable for administration to a particular patient may be determined by a physician considering the patient's age, sex, weight, general medical condition, and the specific condition for which the nanoparticles are being administered and the severity thereof. The physician may also take into account the route of administration, the pharmaceutical carrier, and the nanoparticle's biological activity.

Selection of a suitable pharmaceutical composition will also depend upon the mode of administration chosen. For example, the nanoparticles of the invention may be administered by direct injection or intravenously. In this instance, a pharmaceutical composition comprises the prodrug and/or nanoparticle dispersed in a medium that is compatible with the site of injection.

Prodrugs and/or nanoparticles of the instant invention may be administered by any method. For example, the prodrugs and/or nanoparticles of the instant invention can be administered, without limitation parenterally, subcutaneously, orally, topically, pulmonarily, rectally, vaginally, intravenously, intraperitoneally, intrathecally, intracerbrally, epidurally, intramuscularly, intradermally, or intracarotidly. In a particular embodiment, the prodrug and/or nanoparticle is parenterally. In a particular embodiment, the prodrug and/or nanoparticle is administered orally, intramuscularly, subcutaneously, or to the bloodstream (e.g., intravenously). In a particular embodiment, the prodrug and/or nanoparticle is administered intramuscularly or subcutaneously. Pharmaceutical compositions for injection are known in the art. If injection is selected as a method for administering the prodrug and/or nanoparticle, steps must be taken to ensure that sufficient amounts of the molecules or cells reach their target cells to exert a biological effect. Dosage forms for oral administration include, without limitation, tablets (e.g., coated and uncoated, chewable), gelatin capsules (e.g., soft or hard), lozenges, troches, solutions, emulsions, suspensions, syrups, elixirs, powders/granules (e.g., reconstitutable or dispersible) gums, and effervescent tablets. Dosage forms for parenteral administration include, without limitation, solutions, emulsions, suspensions, dispersions and powders/granules for reconstitution. Dosage forms for topical administration include, without limitation, creams, gels, ointments, salves, patches and transdermal delivery systems.

Pharmaceutical compositions containing a prodrug and/or nanoparticle of the present invention as the active ingredient in intimate admixture with a pharmaceutically acceptable carrier can be prepared according to conventional pharmaceutical compounding techniques. The carrier may take a wide variety of forms depending on the form of pharmaceutical composition desired for administration, e.g., intravenous, oral, direct injection, intracranial, and intravitreal.

A pharmaceutical composition of the invention may be formulated in dosage unit form for ease of administration and uniformity of dosage. Dosage unit form, as used herein, refers to a physically discrete unit of the pharmaceutical composition appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. In a particular embodiment, the prodrugs and/or nanoparticles of the instant invention, due to their long-acting therapeutic effect, may be administered once every 1 to 12 months or even less frequently. For example, the nanoformulations of the instant invention may be administered once every 0.5, 1, 2, 3, 4, 5, 6, 7, 8, 9, 10, 11, 12, 15, 18, 21, 24, or more months. In a particular embodiment, the prodrugs and/or nanoparticles of the instant invention are administered less than once every two months. In a particular embodiment, the prodrugs and/or nanoformulations of the prodrugs are administered once every month, once every two months, particularly once every three months, once every four months, once every five months, once every six months, once every seven months, once every eight months, once every nine months, once every ten months, once every eleven months, once every twelve months, or less frequently.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of nanoparticles may be determined by evaluating the toxicity of the molecules or cells in animal models. Various concentrations of nanoparticles in pharmaceutical composition may be administered to mice, and the minimal and maximal dosages may be determined based on the beneficial results and side effects observed as a result of the treatment. Appropriate dosage unit may also be determined by assessing the efficacy of the nanoparticle treatment in combination with other standard drugs. The dosage units of nanoparticle may be determined individually or in combination with each treatment according to the effect detected.

The pharmaceutical composition comprising the nanoparticles may be administered at appropriate intervals until the pathological symptoms are reduced or alleviated, after which the dosage may be reduced to a maintenance level. The appropriate interval in a particular case would normally depend on the condition of the patient.

The instant invention encompasses methods of treating a disease/disorder comprising administering to a subject in need thereof a pharmaceutical composition comprising a prodrug and/or nanoparticle of the instant invention and, preferably, at least one pharmaceutically acceptable carrier. The instant invention also encompasses methods wherein the subject is treated via ex vivo therapy. In particular, the method comprises removing cells from the subject, exposing/contacting the cells in vitro to the nanoparticles of the instant invention, and returning the cells to the subject. In a particular embodiment, the cells comprise macrophage. Other methods of treating the disease or disorder may be combined with the methods of the instant invention may be co-administered with the pharmaceutical compositions of the instant invention.

The instant also encompasses delivering the nanoparticle of the instant invention to a cell in vitro (e.g., in culture). The nanoparticle may be delivered to the cell in at least one carrier.

### Definitions

The following definitions are provided to facilitate an understanding of the present invention.

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “prodrug” refers to a compound that is metabolized or otherwise converted to a biologically active or more active compound or drug, typically after administration. A prodrug, relative to the drug, is modified chemically in a manner that renders it, relative to the drug, less active, essentially inactive, or inactive. However, the chemical modification is such that the corresponding drug is generated by metabolic or other biological processes, typically after the prodrug is administered.

The term “treat” as used herein refers to any type of treatment that imparts a benefit to a patient afflicted with a disease, including improvement in the condition of the patient (e.g., in one or more symptoms), delay in the progression of the condition, etc. In a particular embodiment, the treatment of a retroviral infection results in at least an inhibition/reduction in the number of infected cells and/or detectable viral levels.

As used herein, the term “prevent” refers to the prophylactic treatment of a subject who is at risk of developing a condition (e.g., HIV infection) resulting in a decrease in the probability that the subject will develop the condition.

A “therapeutically effective amount” of a compound or a pharmaceutical composition refers to an amount effective to prevent, inhibit, treat, or lessen the symptoms of a particular disorder or disease. The treatment of a microbial infection (e.g., HIV infection) herein may refer to curing, relieving, and/or preventing the microbial infection, the symptom(s) of it, or the predisposition towards it.

As used herein, the term “therapeutic agent” refers to a chemical compound or biological molecule including, without limitation, nucleic acids, peptides, proteins, and antibodies that can be used to treat a condition, disease, or disorder or reduce the symptoms of the condition, disease, or disorder.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

The term “antimicrobials” as used herein indicates a substance that kills or inhibits the growth of microorganisms such as bacteria, fungi, viruses, or protozoans.

As used herein, the term “antiviral” refers to a substance that destroys a virus and/or suppresses replication (reproduction) of the virus. For example, an antiviral may inhibit and or prevent: production of viral particles, maturation of viral particles, viral attachment, viral uptake into cells, viral assembly, viral release/budding, viral integration, etc.

As used herein, the term “highly active antiretroviral therapy” (HAART) refers to HIV therapy with various combinations of therapeutics such as nucleoside reverse transcriptase inhibitors, non-nucleoside reverse transcriptase inhibitors, HIV protease inhibitors, and fusion inhibitors.

As used herein, the term “amphiphilic” means the ability to dissolve in both water and lipids/apolar environments. Typically, an amphiphilic compound comprises a hydrophilic portion and a hydrophobic portion. “Hydrophobic” designates a preference for apolar environments (e.g., a hydrophobic substance or moiety is more readily dissolved in or wetted by non-polar solvents, such as hydrocarbons, than by water). “Hydrophobic” compounds are, for the most part, insoluble in water. As used herein, the term “hydrophilic” means the ability to dissolve in water.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

An “antibody” or “antibody molecule” is any immunoglobulin, including antibodies and fragments thereof (e.g., scFv), that binds to a specific antigen. As used herein, antibody or antibody molecule contemplates intact immunoglobulin molecules, immunologically active portions of an immunoglobulin molecule, and fusions of immunologically active portions of an immunoglobulin molecule.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

As used herein, the term “targeting ligand” refers to any compound which specifically binds to a specific type of tissue or cell type, particularly without substantially binding other types of tissues or cell types. Examples of targeting ligands include, without limitation: proteins, polypeptides, peptides, antibodies, antibody fragments, hormones, ligands, carbohydrates, steroids, nucleic acid molecules, and polynucleotides.

The term “aliphatic” refers to a non-aromatic hydrocarbon-based moiety. Aliphatic compounds can be acyclic (e.g., linear or branched) or cyclic moieties (e.g., cycloalkyl) and can be saturated or unsaturated (e.g., alkyl, alkenyl, and alkynyl). Aliphatic compounds may comprise a mostly carbon main chain (e.g., 1 to about 30 carbons) and comprise heteroatoms and/or substituents (see below). The term “alkyl,” as employed herein, includes saturated or unsaturated, straight or branched chain hydrocarbons containing 1 to about 30 carbons in the normal/main chain. The hydrocarbon chain of the alkyl groups may be interrupted with one or more heteroatom (e.g., oxygen, nitrogen, or sulfur). An alkyl (or aliphatic) may, optionally, be substituted (e.g. with fewer than about 8, fewer than about 6, or 1 to about 4 substituents). The term “lower alkyl” or “lower aliphatic” refers to an alkyl or aliphatic, respectively, which contains 1 to 3 carbons in the hydrocarbon chain. Alkyl or aliphatic substituents include, without limitation, alkyl (e.g., lower alkyl), alkenyl, halo (such as F, Cl, Br, I), haloalkyl (e.g., CCl₃ or CF₃), alkoxyl, alkylthio, hydroxy, methoxy, carboxyl, oxo, epoxy, alkyloxycarbonyl, alkylcarbonyloxy, amino, carbamoyl (e.g., NH₂C(═O)— or NHRC(═O)—, wherein R is an alkyl), urea (—NHCONH₂), alkylurea, aryl, ether, ester, thioester, nitrile, nitro, amide, carbonyl, carboxylate and thiol. Aliphatic and alkyl groups having at least about 5 carbons in the main chain are generally hydrophobic, absent extensive substitutions with hydrophilic substituents.

The term “aryl,” as employed herein, refers to monocyclic and bicyclic aromatic groups containing 6 to 10 carbons in the ring portion. Examples of aryl groups include, without limitation, phenyl or naphthyl, such as 1-naphthyl and 2-naphthyl, or indenyl. Aryl groups may optionally include one to three additional rings fused to a cycloalkyl ring or a heterocyclic ring. Aryl groups may be optionally substituted through available carbon atoms with, for example, 1, 2, or 3 groups selected from hydrogen, halo, alkyl, polyhaloalkyl, alkoxy, alkenyl, trifluoromethyl, trifluoromethoxy, alkynyl, aryl, heterocyclo, aralkyl, aryloxy, aryloxyalkyl, aralkoxy, arylthio, arylazo, heterocyclooxy, hydroxy, nitro, cyano, sulfonyl anion, amino, or substituted amino. The aryl group may be a heteroaryl. “Heteroaryl” refers to an optionally substituted, mono-, di-, tri-, or other multicyclic aromatic ring system that includes at least one, and preferably from 1 to about 4, sulfur, oxygen, or nitrogen heteroatom ring members. Heteroaryl groups can have, for example, from about 3 to about 50 carbon atoms (and all combinations and subcombinations of ranges and specific numbers of carbon atoms therein), with from about 4 to about 10 carbons being preferred.

The following example provides illustrative methods of practicing the instant invention and is not intended to limit the scope of the invention in any way.

### Example

Chemically transforming existing native antiretroviral drugs (ARVs) into potent long acting, viral reservoir-targeted agents with extended half-lives provides a paradigm shift in the management of hepatitis B virus (HBV) and other viral infections (McMillan, et al. (2018) AIDS 33(3):585-588; Lin, et al. (2018) Chem. Commun., 54:8371-4; Gu, et al. (2018) PLoS Pathog., 14:e1007061; Zhou, et al. (2018) Biomaterials 151:53-65; Zhou, et al. (2018) Nanomedicine 13(8):871-885; Sillman, et al. (2018) Nat. Commun., 9:443; McMillan, et al. (2017) Antimicrob. Agents Chemother., 62(1):e01316-17; Edagwa, et al. (2018) Nat. Mater., 17:114-6; Edagwa, et al. (2017) Expert Opin. Drug Deliv., 2017:1-11). Creation of hydrophobic and lipophilic prodrug nanocrystals has enabled drug delivery platforms that extend half-lives of both water soluble and hydrophobic ARVs. Long-acting ARVs will positively affect drug adherence and, thereby, reduce viral transmission, prevent new infections, and limit the emergence of drug resistance and systemic toxicities (Spreen, et al. (2013) Curr. Opin. HIV AIDS 8:565-71; Williams, et al. (2013) Nanomedicine 8:1807-13).

Long-acting prodrug nanoformulations of nitazoxanide (NTZ) and tenofovir (TFV) were synthesized. The nanoformulations showed improved drug pharmacokinetics, biodistribution, and HBV suppression in rodents.

Briefly, prodrugs of TFV (M1TAF) and NTZ (M1NTZ) were first synthesized. By example, the acyl ester in NTZ was hydrolyzed to form tizoxanide. Deprotonation of the phenol functional group was performed with a suitable base such as N,N-diisopropylethylamine (DIEA). The resultant was then reacted with either the acyl chloride or activated carboxylic acid of the alkyl fatty acid to arrive at the modified prodrugs. A schematic of a method for the synthesis of the MTZ prodrug is provided:

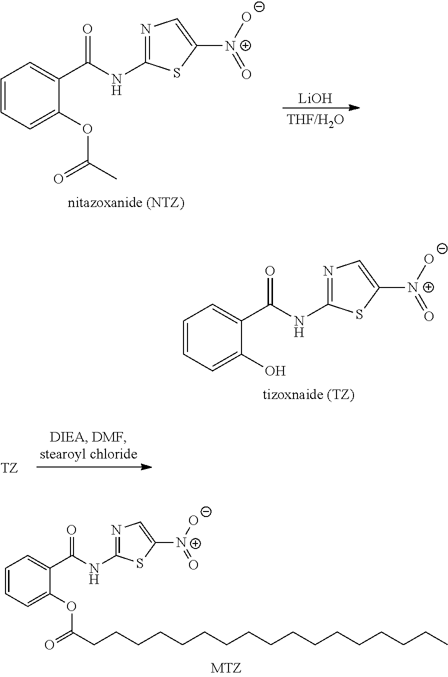

More specifically, the acetyl group in NTZ was hydrolyzed with appropriate reagents. The alcohol anion was then coupled with the fatty acyl chloride or activated carboxylic acid of the alkyl fatty acid to generate the prodrugs. Coupling reagents which can be used to activate the carboxylic acid include, for example, uranium salts, carbodiimide reagents, phosphonium salts, and the like. N,N diisopropylethylamine was used as the base, but other bases could be used. The polar aprotic solvent N,N-dimethylformamide (DMF) was also used in the coupling reaction, but other polar aprotic solvents such as tetrahydrofuran and acetonitrile could be used. The reagents were mixed at 0° C. and gradually warmed to temperature over 12-24 hours. The final compounds were purified on a silica column chromatography and characterized by nuclear magnetic resonance spectroscopy and high-performance liquid chromatography in tandem with mass spectrometry.

Nitazoxanide and tenofovir prodrugs were then loaded into nanoformulations named NM1NTZ and NM1TAF, respectively. Specifically, poloxamer 407-coated nanoformulations were prepared by high-pressure homogenization. Electron microscopy was used to evaluate particle shape and size.

As seen in FIGS. 1A-1C, the chemical modifications altered physicochemical properties of the parent compounds without cytotoxicity (FIG. 1A-C). Briefly, cellular viability following treatment was evaluated by performing a 3-(4,5-dimethylthiazol-2-yl)-2,5-diphenyltetrazolium bromide (MTT) assay. Human MDM plated in 96-well plates at a density of ˜0.08×10⁶ cells per well were treated with various concentrations of drug or nanoparticles for 24 hours. Untreated cells were used as controls. For each group samples were in quadruplets. Cells were washed with PBS and incubated with MTT solution at 37° C. After incubation, MTT solution was removed, and cells were washed with PBS. Then, 200 μL of DMSO was added to each well, and absorbance was measured at 490 nm.

Moreover, the nanoformulations had a uniform particle size of 250-350 nm, a narrow polydispersity index (POI) of <0.2, a negative zeta potential, and a high drug loading capacity (>80%) (FIG. 1D). The high drug loading reduces the volume of injection while the narrow POI indicates formulation homogeneity. Physical and thermal stability of the encapsulated prodrugs at 4°, 25° and 37° C. without particle agglomeration was also observed.

Nanoformulated tenofovir prodrug (NM1TAF) was taken up readily by human monocyte-derived macrophages (MDM) and demonstrated prolonged cell retention with no cytotoxicity. Briefly, human monocytes were plated in a 12-well plate at a density of 1.0×10⁶ cells per well. After 7-10 days of differentiation in the presence of 1000 U/mL recombinant human macrophage colony stimulating factor (MCSF), MDM were treated with prodrug or nanoformulation. Uptake of drug was assessed by measurements of intracellular drug concentrations at various timepoints after treatment. For drug retention studies, cells were treated for 8 hours then washed with PBS and maintained with half-media changes every other day until collection at various timepoints. For both studies, adherent MDM were washed with PBS (3×1 mL), then scraped into 1 mL of fresh PBS, and counted at indicated time points. Cells were pelleted by centrifugation at 4° C. The cell pellet was reconstituted in high performance liquid chromatography (HPLC)-grade methanol and probe sonicated followed by centrifugation. The supernatant was analyzed for drug content using HPLC.

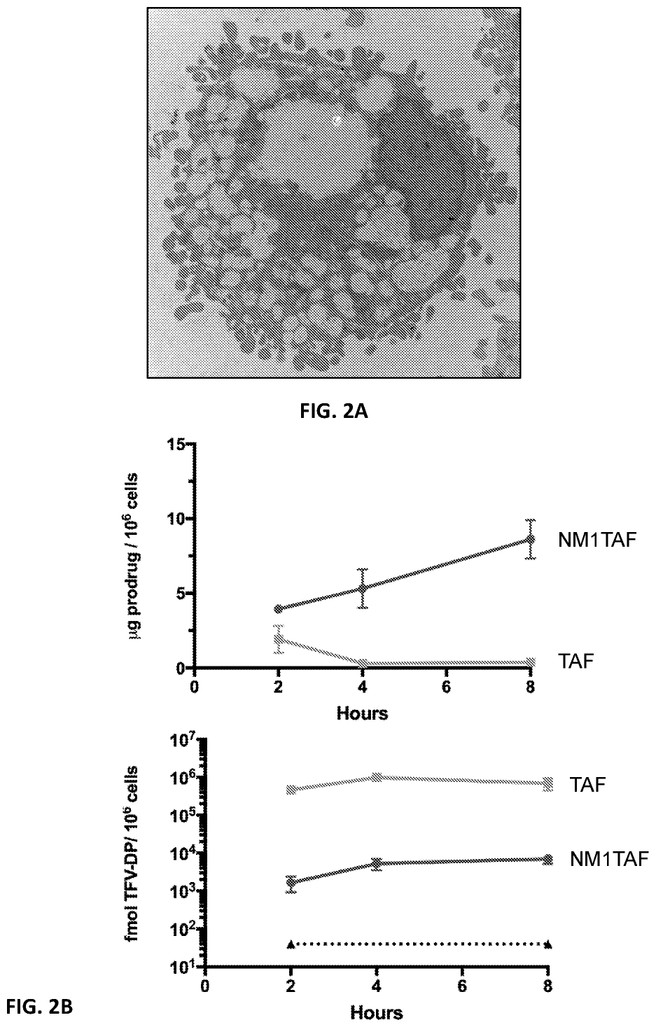

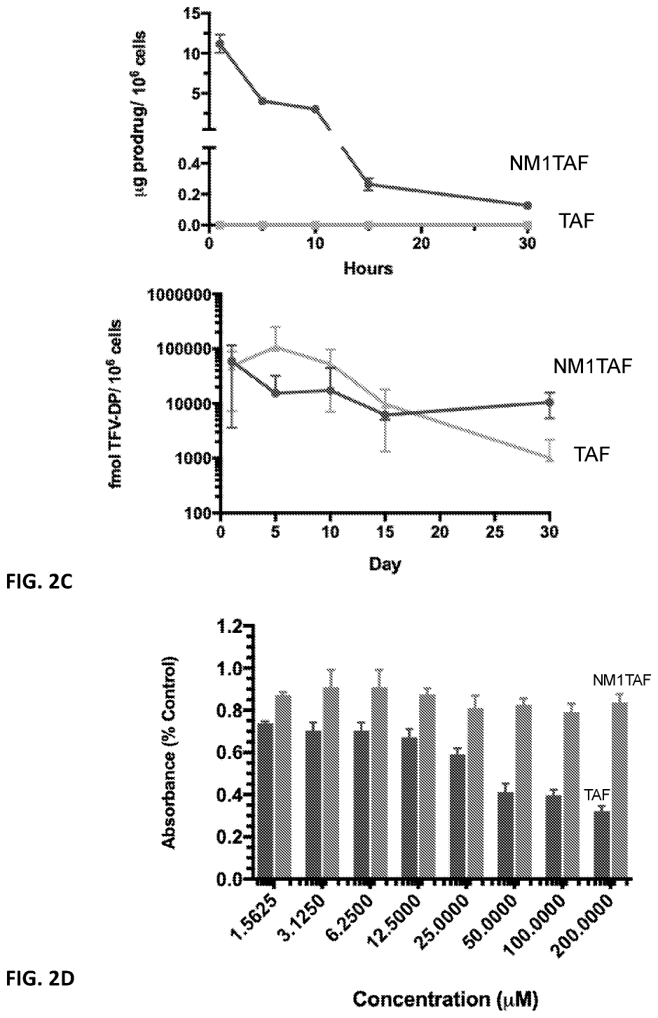

After 8 hours of NM1TAF treatment, about 70% of the macrophage cytoplasm was exchanged with vesicles containing nanoparticles (FIG. 2A). Compared to tenofovir alafenamide (TAF) solution, NM1TAF formulation provided sustained intracellular drug levels with parallel improvements in retention with no toxicity at drug concentrations of ≤200 μM (FIGS. 2B-2D). These data sets demonstrate that modification of tenofovir improves drug cell uptake and retention.

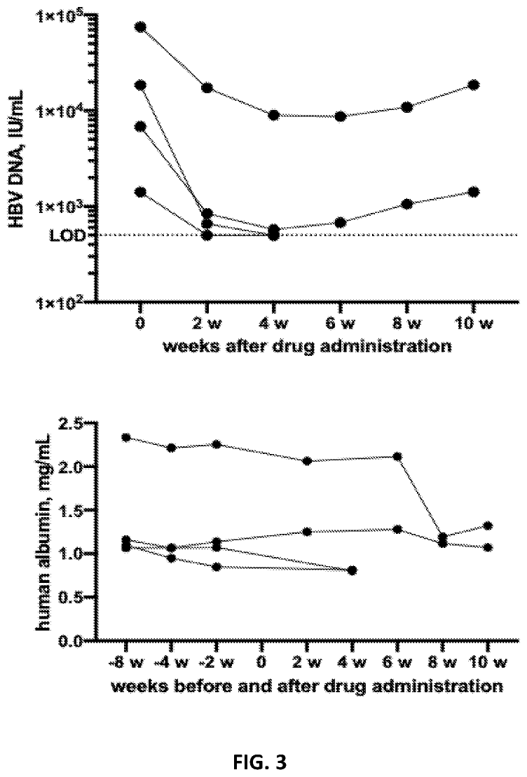

To determine whether long acting prodrug formulations of TFV (NM1TAF) and NTZ (NM1NTZ) could provide improved drug pharmacokinetics and efficacy profiles, their combination was tested in humanized mice models of an HBV infection. In this study, a single dose of NM1TAF+NM1NTZ (75 mg/kg parent drug equivalents for each prodrug formulation) was given. Briefly, TK-NOG mice were transplanted with human hepatocytes, and after confirmation of human albumin (Alb) concentration in peripheral blood, the mice were infected intravenously with patient-derived sera samples containing ˜10⁶ HBV DNA. Upon confirmation of infection via quantitation of HBV DNA in peripheral blood, four animals were administered a single intramuscular dose of a combination therapy consisting of NM1TAF and NM1NTZ formulations at 75 mg/kg native drug equivalents for each drug. HBV DNA and HBsAg in plasma were monitored for four weeks (two animals) and eight weeks (two animals). Notably, the combination therapy reduced HBV DNA in plasma to undetectable levels in two of the animals at four weeks (sacrificed for tissue drug and viral load analyses) post drug treatment, without loss of human cells (FIG. 3). The other two animals demonstrated more than a log decrease in plasma viral load at four weeks and were monitored for four additional weeks and sacrificed. These data sets demonstrate that NM1TAF and NM1NTZ lead to effective once/month or longer dosing intervals to provide sustained control of viral replication.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A prodrug of a thiazolide, wherein said prodrug comprises an ester moiety, wherein said ester moiety comprises a hydrophobic and/or lipophilic moiety,
or a pharmaceutically acceptable salt thereof.

2. The prodrug of claim 1, wherein said ester moiety is at the 2-position of the benzene of the thiazolide.

3. The prodrug of claim 1, wherein the hydrophobic and/or lipophilic moiety is a saturated or unsaturated linear or branched aliphatic chain.

4. The prodrug of claim 3, wherein the aliphatic chain is 4 to 24 carbon atoms in length; and/or
wherein the aliphatic chain comprises one or more heteroatoms, an aromatic moiety optionally substituted with one or more heteroatoms, and/or one or more amino acids.

5. (canceled)

6. The prodrug of claim 1, wherein said thiazolide is selected from the group consisting of tizoxanide, nitazoxanide, haloxanide, RM-4832, RM-4848, RM-4850, RM-4851, RM-4852, and RM-4863.

7. The prodrug of claim 1 having the formula: 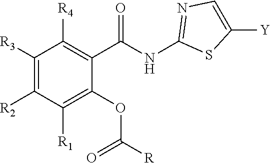
wherein R is said hydrophobic and/or lipophilic moiety;
wherein R₁-R₄ are independently selected from the group consisting of hydrogen, hydroxyl, alkoxy, alkyl, and halogen;
and wherein Y is selected from the group consisting of hydrogen, nitro, sulfonyl, hydroxyl, alkoxy, alkyl, and halogen;
or a pharmaceutically acceptable salt or stereoisomer thereof.

8. The prodrug of claim 7, wherein at least three of R₁-R₄ are hydrogen; and/or
wherein R is a saturated or unsaturated linear or branched aliphatic chain.

9. (canceled)

10. The prodrug of claim 1 having the formula: 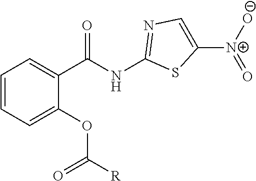
wherein R a hydrophobic and/or lipophilic moiety,
or a pharmaceutically acceptable salt or stereoisomer thereof.

11. The prodrug of claim 10, wherein R is a saturated or unsaturated linear or branched aliphatic chain.

12. The prodrug of claim 11, wherein said aliphatic chain is 4 to 24 carbon atoms in length;
wherein R is the side chain of a fatty acid; and/or
wherein R is a saturated linear hydrocarbon chain, optionally wherein said hydrocarbon chain is 15 to 19 carbons in length.

13-15. (canceled)

16. The prodrug of claim 1 having the formula: 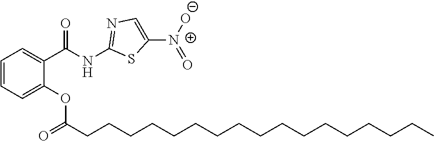 [SMILES: CCCCCCCCCCCCCCCCCC(=O)Oc1ccccc1C(=O)Nc1ncc([N+](=O)[O-])s1]
or a pharmaceutically acceptable salt or stereoisomer thereof.

17. The prodrug of claim 1, wherein said prodrug comprises a dimer of a first thiazolide and a second thiazolide, wherein said first and second thiazolides each comprise an ester moiety, and wherein the ester moiety of the first thiazolide is covalently attached to the ester moiety of the second thiazolide by a hydrophobic and/or lipophilic moiety,
or a pharmaceutically acceptable salt thereof.

18. The prodrug of claim 17, wherein said first and second thiazolides are the same or wherein said first and second thiazolides are different.

19. (canceled)

20. The prodrug of claim 17, wherein said ester moieties are at the 2-positions of the benzenes of the first and second thiazolides.

21. The prodrug of claim 17, wherein the hydrophobic and/or lipophilic moiety is a saturated or unsaturated linear or branched aliphatic chain.

22. The prodrug of claim 21, wherein the aliphatic chain is 4 to 24 carbon atoms in length; and/or
wherein the aliphatic chain comprises one or more heteroatoms, an aromatic moiety optionally substituted with one or more heteroatoms, and/or one or more amino acids.

23. (canceled)

24. The prodrug of claim 17, wherein said first and second thiazolides are selected from the group consisting of tizoxanide, nitazoxanide, haloxanide, RM-4832, RM-4848, RM-4850, RM-4851, RM-4852, and RM-4863.

25. The prodrug of claim 17 having the formula: 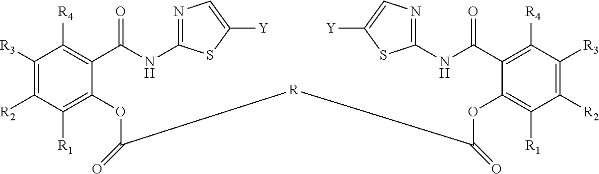
wherein R is said hydrophobic and/or lipophilic moiety;
wherein R₁-R₄ are independently selected from the group consisting of hydrogen, hydroxyl, alkoxy, alkyl, and halogen;
and wherein Y is selected from the group consisting of hydrogen, nitro, sulfonyl, hydroxyl, alkoxy, alkyl, and halogen;
or a pharmaceutically acceptable salt or stereoisomer thereof.

26. The prodrug of claim 25, wherein at least three of R₁-R₄ are hydrogen; and/or
wherein R is a saturated or unsaturated linear or branched aliphatic chain.

27. (canceled)

28. The prodrug of claim 17 having the formula: 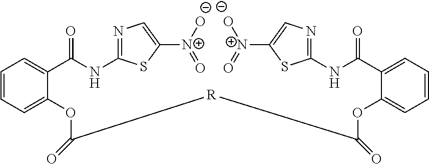
wherein R a hydrophobic and/or lipophilic moiety,
or a pharmaceutically acceptable salt or stereoisomer thereof.

29. The prodrug of claim 28, wherein R is a saturated or unsaturated linear or branched aliphatic chain.

30. The prodrug of claim 29, wherein said aliphatic chain is 4 to 24 carbon atoms in length;
wherein R is the side chain of a fatty acid; and/or
wherein R is a saturated linear hydrocarbon chain, optionally wherein said hydrocarbon chain is 15 to 19 carbons in length.

31-33. (canceled)

34. A nanoparticle comprising at least one prodrug of claim 1 and at least one polymer or surfactant.

35. The nanoparticle of claim 34, wherein said prodrug and/or nanoparticle is crystalline.

36. The nanoparticle of claim 34, wherein said polymer or surfactant is an amphiphilic block copolymer;
wherein said amphiphilic block copolymer comprises at least one block of poly(oxyethylene) and at least one block of poly(oxypropylene); and/or
wherein the polymer or surfactant is poloxamer 407.

37-38. (canceled)

39. The nanoparticle of claim 34, wherein said nanoparticle further comprises a polymer or surfactant linked to at least one targeting ligand.

40. The nanoparticle of claim 34, wherein the diameter of the nanoparticle is about 100 nm to 1 μm.

41. A composition comprising at least one nanoparticle of claim 34 and at least one pharmaceutically acceptable carrier.

42. A composition comprising at least one prodrug of claim 1 and at least one pharmaceutically acceptable carrier.

43. A method for treating, inhibiting, and/or preventing a disease or disorder in a subject in need thereof, said method comprising administering to said subject a prodrug of claim 1.

44. The method of claim 43, wherein the disease or disorder is a viral infection, bacterial infection, parasitic infection, cancer, pain, neurodegenerative disease, or aging-related disease.

45. (canceled)

46. The method of claim 44, wherein the viral infection is selected from the group consisting of Hepatitis A infections, Hepatitis B infections, Hepatitis C infections, HIV infections, Influenza infections, Rhinovirus infections, Adenovirus infections, Parainfluenza infections, Rotavirus infections, Norovirus infections, coronavirus infections, and respiratory syncytial virus infections; optionally wherein said viral infection is an HIV infection, coronavirus infection, or HBV infection.

47. The method of claim 44, further comprising administering a further therapeutic agent.

48. The method of claim 47, wherein said further therapeutic agent is a LASER ART and/or ProTide LASER ART; and/or
wherein said further therapeutic agent is tenofovir prodrug.

49. (canceled)

50. (canceled)
